(19) United States
(12) Patent Application Publication
(10) Pub. No.: US 2008/0177210 A1
(43) Pub. Date: Jul. 24, 2008

(54) ORTHOTIC DEVICE FOR AN ANIMAL

(76) Inventor: Nicole Suzanne McDevitt Larson, Bellingham, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/867,576

(22) Filed: Oct. 4, 2007

Related U.S. Application Data
(60) Provisional application No. 60/828,482, filed on Oct. 6, 2006.

(51) Int. Cl. A61F 5/00; A01K 27/00

(52) U.S. Cl. 602/23; 119/856; 602/2

(57) ABSTRACT

An orthotic device for a leg of an animal is disclosed. The orthotic device includes a flexible base, which is conformable to an exterior surface of the leg, and a stabilizing member, which can be selectively formed to substantially match an exterior contour of the leg. The stabilizing member can be attached to and removed from the flexible base.

### CROSS-REFERENCES

This application claims the benefit of the filing date of U.S. Provisional Patent Application Ser. No. 60/828,482, filed on Oct. 6, 2006, the disclosure of which is hereby expressly incorporated by reference.

### FIELD OF THE DISCLOSURE

Embodiments of the present disclosure relate generally to orthotic devices for animals and, more particularly, to a knee brace.

### BACKGROUND

Domesticated animals, such as dogs, frequently suffer from chronic knee pain and joint damage. The most commonly suffered knee injury is a ruptured cruciate ligament, which often requires surgical repair. An orthotic device, such as a knee brace, speeds the recovery process and alleviates pain by providing stability and control to injured or healing joints. Immediate support to the injured knee is essential in preventing further damage to the joint.

A method for fabricating a known knee brace requires creating a mold of the affected leg. The mold is sent to a manufacturer that uses the mold to fabricate a knee brace customized to fit the leg from which the mold was made. Although a customized knee brace fabricated in this manner provides suitable fit and function, such a device is relatively expensive and often requires several months to produce. Thus, there is a need for a more cost effective orthotic device that can be quickly adapted to fit the leg of a particular animal.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

An orthotic device for a leg of an animal is provided. The orthotic device includes a flexible base conformable to an exterior surface of the leg and a stabilizing member. The stabilizing member being selectively formable to substantially match an exterior contour of the leg. The stabilizing member is removably attachable to the flexible base.

The orthotic device is suitable for use of a variety of different sizes of animals due the use of a flexible base garment in conjunction with one or more moldable stabilizer members. The disclosed embodiment includes four stabilizer members that can be removed or switched out depending on the needs of the animal. The orthotic device is fully adjustable for each body size and shape and a harness can be used to aid in keeping the device in the correct location. The disclosed orthotic device can be used for either rear leg and can be easily adapted to be used for the front legs as well. Retention means are optionally provided to secure additional therapeutic devices, such as heat packs and interferential stimulators to the orthotic device.

### DESCRIPTION OF THE DRAWINGS

The foregoing aspects and many of the attendant advantages of this invention will become better understood by reference to the following detailed description, when taken in conjunction with the accompanying drawings, wherein:

FIG. 1 is an isometric view of an orthotic device constructed in accordance with one embodiment of the present disclosure, wherein the orthotic device is stabilizing the knee joint on the hind leg of a dog;

FIG. 2 is a side planar view of the orthotic device shown in FIG. 1; and

FIG. 3 is a side view of the orthotic device shown in FIG. 1, showing details of the orthotic device relative to the knee joint of the dog.

### DETAILED DESCRIPTION

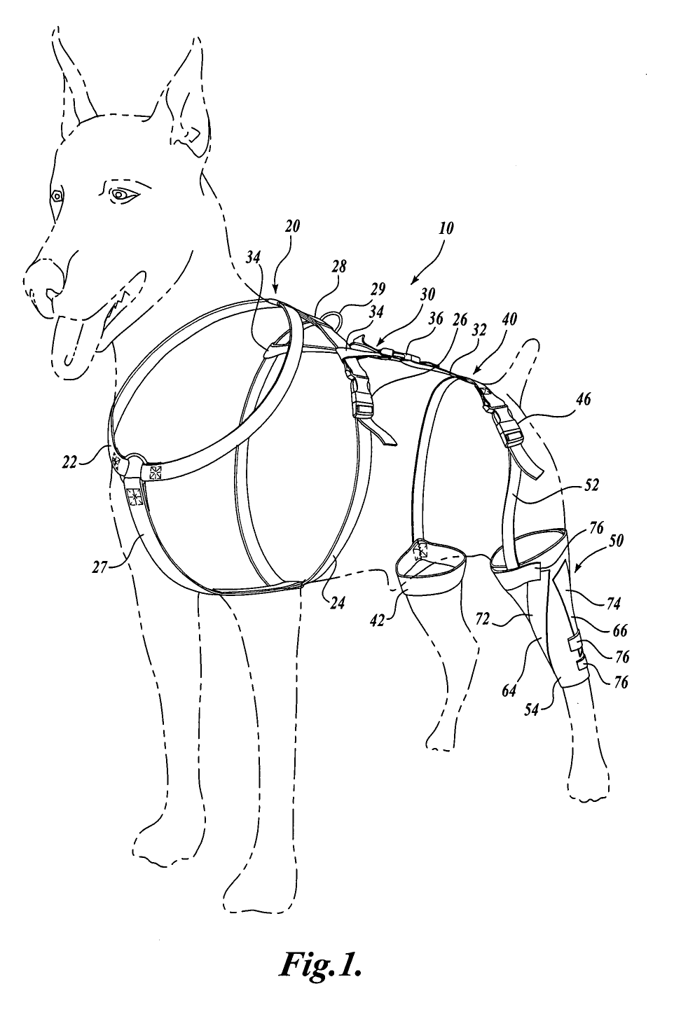

An orthotic device 10 constructed in accordance with one embodiment of the present disclosure may be best understood by referring to FIG. 1. The orthotic device 10 includes a harness 20, a longitudinal bracing assembly 30, a lateral bracing assembly 40, and a stabilizer assembly 50. Although illustrated as being used worn to stabilize the left hind leg of a dog, it should be understood that the scope of the orthotic device is not intended to be so limited, and that orthotic devices for us on other animals, such as horses, cats, etc., are also within the scope of the present disclosure. As such, the use of the term “dog” is intended to be illustrative and not limiting. It should also be appreciated that an orthotic device in accordance with the disclosed subject matter is not limited to any particular leg of an animal, but may be configured to provide support to any of an animal's legs, including front and hind legs, as well as left and right legs.

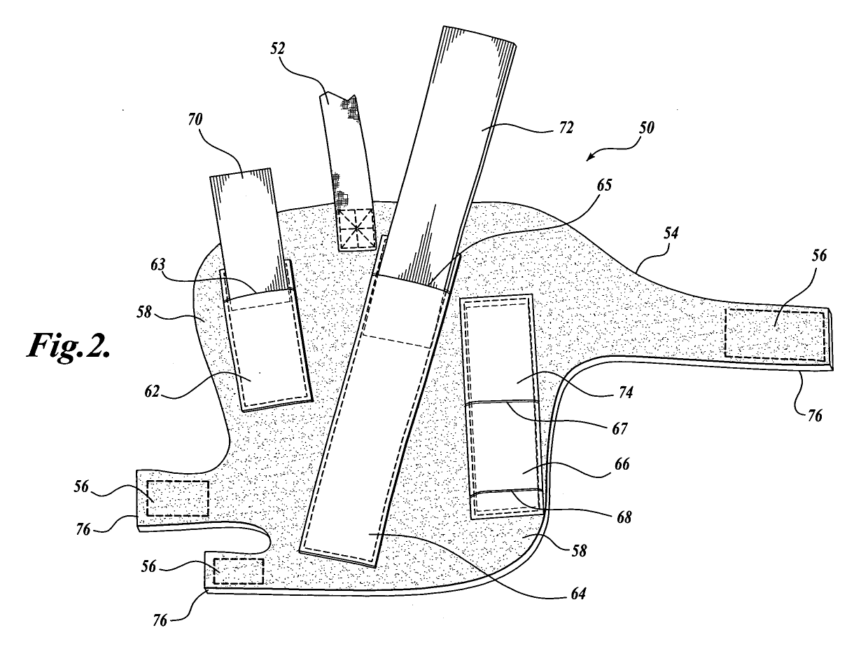

As best shown in FIG. 2, an embodiment of the stabilizer assembly 50 includes a flexible base 54 having a plurality of integral straps 76 extending laterally therefrom. The flexible base 54 is sized to engage an area around the knee, both above and below, as well as circumferentially, that is sufficient to enable the stabilizer assembly 50 to immobilize the knee. Each integral strap 76 has a length such that when the flexible base 54 is wrapped around the knee, the integral strap 76 overlaps the side of the flexible base 54 opposite to the side from which the integral strap 76 extends. The flexible base 54 preferably includes a synthetic rubber material, which is currently used to manufacture known flexible knee braces; however, any material having suitable strength, durability, and flexibility may be used. Further, although the straps 76 are illustrated as being integral to the flexible base 54, it should be understood that straps may be formed separate from the flexible base 54 and attached by suitable means. In will be readily apparent to one of skill in the art that straps 76 formed separate from the flexible base 54 need not be made from the same material as the base 54, but can comprise any material of suitable strength and flexibility, such as, for example, nylon. In addition, the number, shape, and orientation of the straps 76 may be adapted according to the size and type of animal for which the stabilizer assembly 50 is intended.

A hook portion 56 of a hook and latch fastener system is attached to one side of the flexible base 54 at each integral strap 76. The corresponding latch portion 58 of the hook and latch fastener system is attached to at least a portion of the opposite side of the flexible base 54. The latch portion 58 is positioned so that when the flexible base 54 is wrapped around the leg, the hook portion 56 attached to each integral strap 76 engages the latch portion 58, thereby securing the strap 76 to the flexible base 54. This in turn secures the stabilizer assembly 50 to the leg of the dog. A sufficient amount of the hook portion 56 and the latch portion 58 are provided so that each integral strap may be secured to the opposite side of the base 54 in a number of different positions, thereby enabling the flexible base 54 to be adjusted for use on a variety of dogs having different sizes.

It should be understood that the hook portion 56 and the latch portion 58 of the hook and latch fastener system can be reversed so that the latch portion 58 is attached to the integral straps and the hook portion is attached to the opposite side of the flexible base 54. In should also be apparent that alternative means can be used to secure opposite ends of the flexible base 54 to each other, including snaps, buttons, laces, zippers, etc. In still another alternative embodiment, the flexible base 54 is formed to have a closed cross-section so that the stabilizer assembly 50 is positioned on the knee by sliding the stabilizer assembly 50 over the paw of the dog and up the leg until the stabilizer assembly 50 is properly positioned.

Still referring to FIG. 2, a plurality of pockets 62, 64, and 66 are located on the flexible base 54 to contain the conformable stabilizers 70, 72, and 74, respectively, which will be described in detail later. Each pocket is formed of a flexible material having suitable strength to hold a conformable stabilizer in place and to resist premature wear. In one non-limiting embodiment, the flexible material is ballistic nylon, although other suitable materials will be apparent to those of skill in the art. The flexible material is secured to the base 54 with stitching, adhesives or other suitable means. In one alternate embodiment, the pockets are integrally formed in the flexible base 54.

Each pocket 62, 64, and 66 has at least one slot 63, 65, 67, and 68 through which a conformable stabilizer 70, 72, and 74, can be passed to insert the stabilizer into the pocket. Optionally, as with pocket 66, multiple slots 67 and 68 can be included in a single pocket to allow for multiple conformable stabilizers to be inserted into the single pocket. Inserting multiple stabilizers into a single pocket provides a stabilization area with flexible regions between the individual stabilizers. Additional pockets may optionally be included to retain various therapeutic devices, such as heat packs, interferential stimulators, and other devices that are well know in the art.

Conformal stabilizers, or stabilizing members, 70, 72, and 74 are manufactured from a low temperature thermoplastic material. For a given thermoplastic, the glass transition temperature (Tg) is defined as the temperature at which that thermoplastic begins to soften. Preferably, the conformal stabilizers are formed from a thermoplastic having a Tg less than 180° F. Because the Tg is ideally less than 180° F., a preferred thermoplastic material will heat up and begin to soften when placed in boiling water.

The conformal stabilizers 70, 72, and 74 are initially manufactured in a generally flat, rectangular shape. The flat stabilizers 70, 72, and 74 are then individually reformed to the match the contours of the particular dog's leg on which they will be used. The stabilizers 70, 72, and 74 are reformed by placing them in hot water to heat the stabilizers to a temperature greater than the Tg of the thermoplastic material. When stabilizers 70, 72, and 74 reach a temperature greater than the Tg of the thermoplastic, the stabilizers soften to the point that they can be reformed with hand pressure. Each stabilizer is customized for a particular dog by holding the softened stabilizer against the appropriate region of the dog's leg with sufficient pressure to conform the stabilizer to the leg. As the stabilizer cools to ambient temperature, the temperature of the stabilizer drops below the Tg of the thermoplastic material, and the stabilizer regains its previous stiffness. Having regained its stiffness, the stabilizer maintains a shape that conforms to the surface of the dog's leg. It should be understood that alternate heating methods, such as an oven or a hot air gun, may be employed to heat the stabilizers without departing from the scope of the disclosure.

In order to prevent burns or discomfort during the forming process, a flexible material is placed between the heated stabilizer and the dog's leg to act as a thermal barrier between the stabilizer and the leg. In one embodiment, the stabilizer assembly 50 placed on the dog's leg without the stabilizers 70, 72, and 74 being installed. The heated stabilizers are then formed by holding the heated stabilizers against the outside of the flexible base 54. Correct placement of the stabilizers is ensured by holding each stabilizer over the appropriate stabilizer pocket 62, 64, and 66.

In addition to positioning multiple stabilizers on the flexible base 54 to create areas of increased and reduced stiffness in the stabilizer assembly, a single stabilizer may be formed to have variable thickness along the length of the stabilizer. For example, the stabilizer may be formed to include a laterally oriented region of reduced thickness, thereby creating a region of reduced stiffness. The area of reduced stiffness can act as an integral hinge that provides increased flexibility of the stabilizer assembly 50 at a desired location. It will be apparent to one skilled in the art that various combinations of the number and locations of pocket openings are possible without departing from the spirit and scope of the invention. Likewise, it will also be apparent to one skilled in the art that variations in the number and stiffness characteristics of stabilizer, including the addition of previously mentioned integral hinges, are possible without departing from the spirit and scope of the disclosure.

Referring back to FIG. 1, a lateral bracing assembly 40 includes an attachment strap 52. A loop 42 is attached to a first end of the attachment strap, and the second end of the attachment strap 52 is secured to an upper portion of the stabilizer assembly 50. The attachment strap 52 and loop 42 are preferably made from a soft, flexible material such as nylon strapping or a flexible synthetic rubber, but is should be appreciated that any suitable material may be used. When the stabilizer assembly 50 is positioned on a dog's leg, the loop 42 of the lateral bracing assembly 40 is placed around the leg opposite to the leg being stabilized by the stabilizer assembly 50, and the attachment strap extends laterally across the dog's back, forward of the tail. As a result, the stabilizer assembly 50 is restrained from vertical movement by a combination of the natural taper of the dog's leg and the generally vertical and slightly forward restraining force provided by the attachment strap 52. A buckle 46 may be included at an optional break in the attachment strap 52 to facilitate putting the lateral bracing assembly 50 on the dog and also to provide adjustability for the length of the attachment strap 52.

A longitudinal bracing assembly 30 is attached to the lateral bracing assembly 40 to restrain the stabilizing assembly 50 against longitudinal movement and to secure the lateral bracing assembly 40 in a generally fixed position relative to the dog. The longitudinal bracing assembly 30 includes a longitudinal strap 32 attached at one end to the lateral bracing assembly 40 and attached at the other end to a pair of harness attachment straps 34. As shown in FIG. 1, one end of each harness attachment strap 34 is secured to the end of the longitudinal strap form a “Y” shape. The other end of each of the harness attachment straps 34 is secured to a harness 20, described later, which resists longitudinal movement of the longitudinal strap 32. The longitudinal bracing assembly 30 is constructed from materials similar to those used for the lateral bracing assembly 40. Also, like the attachment strap 52 of the lateral bracing assembly 40, the longitudinal strap 32 may include a buckle 26 at an optional break in the longitudinal strap 32 to facilitate putting the longitudinal bracing assembly 30 on the dog, and also to provide adjustability for the length of the longitudinal strap 32.

The harness, or device positioning means, 20 provides an stabilizer member attachment feature for securing the forward end of the longitudinal bracing assembly 30 in a fixed position relative to the dog. The harness 20 includes a forward harness loop 22, sized to fit around the neck of the dog so that the loop rests on the dog's shoulders, and a rear harness loop 24, sized to fit around the chest of the dog, just rear of the front legs. A dorsal strap 28 is positioned longitudinally along the dog's back and is attached at one end to an upper portion of the forward harness loop 22 and at the other end to an upper portion of the rear harness loop 24. A ventral strap 27 is positioned longitudinally along the dog's chest, between the front legs. A first end of the ventral strap 27 is secured to a lower portion of the forward harness loop 22, and a second end of the ventral strap 27 is secured to a lower portion of the rear harness loop 24. An optional ring 29 is attached to the upper portion of the rear harness loop 24 to provide an attachment point for a leash.

The harness 20 is constructed from materials similar to those used for the lateral bracing assembly 40 and the longitudinal bracing assembly 30. As shown in FIG. 1, the rear harness loop 24 includes a buckle 26 at an optional break in the rear harness loop 24 to facilitate putting the harness 20 on the dog, and also to provide adjustability of the harness 20. It should be appreciated that the illustrated harness 27 is intended to be an exemplary embodiment and should not be considered limiting. Any known animal harness 20 may be utilized without departing from the scope of the disclosed subject matter. It should be further appreciated that a number of variations in the configuration of the lateral bracing assembly 40, the longitudinal bracing assembly 30, and the harness 20 are within the scope of the present disclosure.

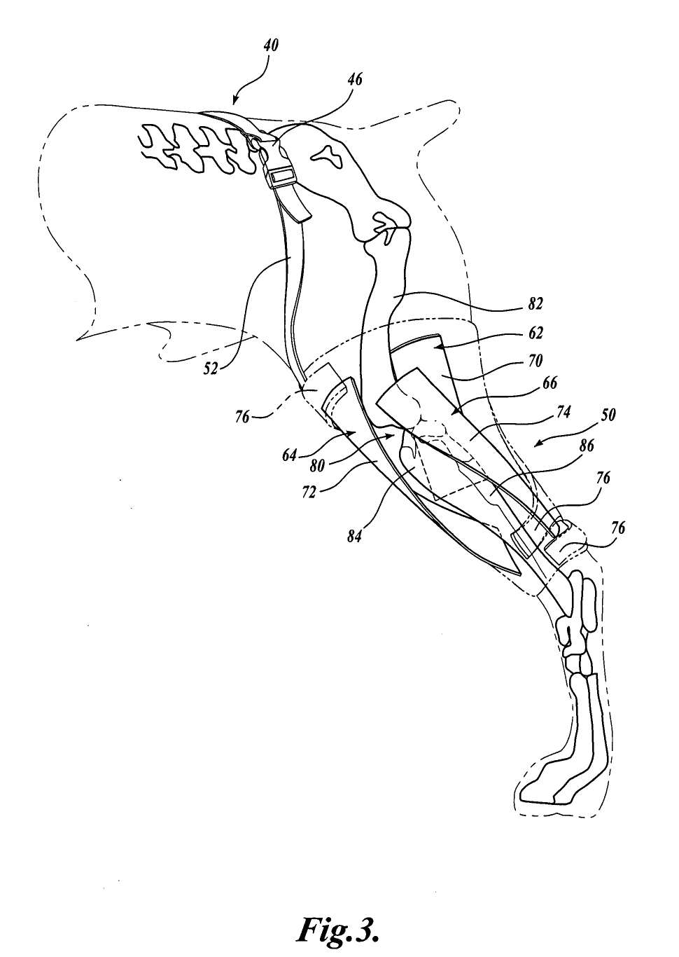

Referring to FIG. 3 the stabilizer assembly 50 is positioned over a knee joint 80 to limit the relative movement of a dog's leg bones by stabilizing the knee joint 80. More specifically, stabilizing the knee joint 80 provides for constrained relative movement between the femur 82 and the tibia 84. Also shown is the fibula 86, which attaches to the tibia 84 adjacent to knee joint 80. Although not depicted in FIG. 3, the knee joint 80 further includes the anterior (cranial) cruciate ligament and the posterior (caudal) cruciate ligament, which in combination pivotally couple the femur 82 and the tibia 84. In operation, the forces transmitted between the femur 82 and the tibia 84 result in significant internal stresses in the cruciate ligaments, especially during abnormal relative twisting between the femur 82 and the tibia 84 due to the crossing orientations of the cruciate ligaments.

The stabilizer assembly 50 interacts with exterior surface of the leg in a way that limits movement between the femur 82 and the tibia 84, as well as providing an alternate load path for transverse and twisting forces that would ordinarily strain the cruciate ligaments. When worn by the dog, the stabilizer assembly 50 is in contact with the exterior surface of the leg and is stiffened by conformal stabilizers 70, 72, and 74, which are in held in pockets 62, 64, and 66 respectively. As a result, the constraint of the dog's leg provided by stabilizer assembly 50 serves to reduce the relative movement between the femur 82 and the tibia 84, thereby limiting the strain in the cruciate ligaments.

While illustrative embodiments have been illustrated and described, it will be appreciated that various changes can be made therein without departing from the spirit and scope of the disclosure.

1. An orthotic device for a leg of an animal, the orthotic device comprising:
a flexible base conformable to an exterior surface of the leg; and
a stabilizing member, the stabilizing member being selectively formable to substantially match an exterior contour of the leg, the stabilizing member being removably attachable to the flexible base.

2. The orthotic device of claim 1, wherein the stabilizing member comprises a low-temperature thermoplastic material.

3. The orthotic device of claim 2, wherein the low-temperature thermoplastic material has a glass transition temperature of less than 180° F.

4. The orthotic device of claim 1, wherein the flexible base includes a pocket adapted to accept the stabilizing member and retain the stabilizing member in a fixed position relative to the flexible base.

5. The orthotic device of claim 1 wherein the device further comprises a device positioning means for maintaining the device in a predetermined position relative to the leg.

6. The orthotic device of claim 5, wherein the device positioning means comprises a first strap positioned laterally across a dorsal portion of the animal, a first end of the first strap being attached at one end to the flexible base, and a second end of the first strap being secured relative to the animal.

7. The orthotic device of claim 6, wherein the device positioning means further comprises a second strap positioned longitudinally along a dorsal portion of the animal, a first end of the second strap being attached to a central portion of the first strap, and a second end of the first strap being secured relative to the animal.

8. The orthotic device of claim 1, further comprising a retention feature for retaining a therapeutic device.

9. The orthotic device of claim 8, wherein the therapeutic device is selected from the group consisting of a heat pack and an interferential stimulator.

10. An orthotic device for stabilizing a leg of an animal, the orthotic device comprising:
a flexible base conformable to an exterior surface the leg;
a stabilizer member, the stabilizer member being selectively moldable to substantially match an exterior contour of the leg; and
a stabilizer member attachment feature for securing the stabilizer member relative to the flexible base.

11. The orthotic device of claim 10, wherein the stabilizer member comprises a low-temperature thermoplastic material.

12. The orthotic device of claim 11, wherein the low-temperature thermoplastic material has a glass transition temperature of less than 180° F.

13. The orthotic device of claim 10, further comprising a pocket disposed on the flexible base, the pocket being adapted to retain the stabilizer member when the stabilizer member is located therein.

14. The orthotic device of claim 10, further comprising a harness for restraining the orthotic device against moving relative to the leg.

15. The orthotic device of claim 14, wherein the harness comprises a first strap positioned laterally across a dorsal portion of the animal, a first end of the first strap being attached at one end to the flexible base, and a second end of the first strap being secured relative to the animal.

16. The orthotic device of claim 15, wherein the harness further comprises a second strap positioned longitudinally along a dorsal portion of the animal, a first end of the second strap being attached to a central portion of the first strap, and a second end of the first strap being secured relative to the animal.

17. The orthotic device of claim 10, further comprising a retention feature for retaining a therapeutic device.

18. The orthotic device of claim 17, wherein the therapeutic device is selected from the group consisting of a heat pack and an interferential stimulator.
